(19) United States
(12) Patent Application Publication
(10) Pub. No.: US 2016/0074510 A1
(43) Pub. Date: Mar. 17, 2016

(54) METHOD AND DEVICE TO DELIVER ANTIFIBRINOLYTIC DRUGS TO IMPEDE TUMOR GROWTH AND INCREASE THE RADIO SENSITIVITY OF CANCEROUS TISSUE

(71) Applicant: Frank Murdock, Copell, TX (US)

(72) Inventor: Frank Murdock, Copell, TX (US)

(21) Appl. No.: 14/848,923

(22) Filed: Sep. 9, 2015

Related U.S. Application Data
(60) Provisional application No. 62/050,247, filed on Sep. 15, 2014.

(51) Int. Cl. A61K 41/00

(52) U.S. Cl. CPC A61K 41/0038

(57) ABSTRACT

Antifibrinolytic agents/drugs are applied to a cancerous area to impede tissue growth and increase the radio sensitivity of cancerous tissue. Various techniques are described for administering the antifibrinolytic agent.

### CROSS-REFERENCE TO RELATED APPLICATION

This application is based on provisional application Ser. No. 62/050,247, filed Sep. 15, 2014, all of the details of which are incorporated herein by reference thereto.

### FIELD OF INVENTION

This invention relates to devices, methods and pharmacologic drugs and preparations for the inhibition of the growth of tumors and cancerous cells. In particular, the invention can act acutely and/or over an extended period of time to utilize/improve on the drug and preparation's anti-cancer/tumor performance, anti-inflammatory capacity as well as its ability to enhance the radio sensitivity of the effected tissue.

### BACKGROUND OF INVENTION

In the field of cancer treatments, several approaches are utilized; delivery of different pharmacologic compounds (such as chemotherapy), surgical excision and radiation treatments are current practice. These treatments are designed to remove well defined tumors and remove and mitigate the growth of diffuse and/or metastatic disease. In practice anti-cancer/tumor drugs are typically delivered systemically or locally and act to limit cancerous cell growth by encouraging cell death via toxicity. Common methods of cancer treatment typically involve a surgical excision of the tumor or cancerous area followed by subsequent treatments involving chemotherapy and/or radiation. It has long been the goal of cancer treatments to utilize surgical excision followed by direct local administration of an active anti-tumor agent such as the Gliadel wafer that can improve on the local control of the disease. Further it has long been a goal that the active anti-tumor agent might have immediate local anti-tumor impact and/or a controlled sustained release to extend the activity of the anti-cancer compound. Finally it has been a goal of cancer treatments to improve on radiation regimens by delivering a lethal radiation dose to the target area while minimizing the destructive effects of radiation to the surrounding healthy tissue. An improved approach to treating the disease would be to inhibit the spread of cancerous cells and to increase the sensitivity of the targeted tissue to radiation so that dosages could be optimized and preferentially delivered (for example pre, intra, and/or post operatively) to a prescribed location.

Antifibrinolytic compounds such as tranexamic acid, aminoacaproic acid, aprotinin and new strategies such as kunitz type inhibitor polypeptide and similar polypeptide structures such as KD1 are typically delivered systemically (iv and/or oral) to control excessive bleeding primarily due to their ability to inhibit plasmin activation. U.S. Pat. No. 7,022,125 (all of the details of which are incorporated herein by reference thereto) discloses applying an antifibrinolytic agent to a substrate to effect hemostasis of a bleeding wound. Over the years they have also been briefly illustrated in the literature to potentially inhibit tumor growth and/or metastasis (possibly as a result of their ability to alter fibrin degradation which effects fibrinolytic pathways and tumor blood supply). In addition, antifibrinolytic agents have been shown to have an anti-inflammatory protective effect in tissue and to increase the radiosensitivity of some tumor cells. However, to date no surgical devices or methods have been described to utilize the anti-cancer, anti-inflammatory functionality of antifibrinolytic agents to improve on cancer treatments.

### SUMMARY OF INVENTION

In accordance with this invention tumor growth and/or spread is impeded and radio sensitivity of cancerous tissue is increased by administering an antifibrinolytic agent to the cancerous area to restrain fibrinolytic activity in the area.

### DETAILED DESCRIPTION

The present invention is based upon the realization that the body's response to trauma/surgery/disease involves the activation (local or otherwise) of the fibrinolytic process. In its broad aspect the invention is practiced by countering the fibrinolytic process through the administration of an antifibrinolytic agent to play an important role in improving treatments for these situations. In the specific situation of cancerous growths, that antifibrinolytic drugs if applied effectively (topically and/or systemically) in a manner which can act immediately and/or which extends their activity in the affected area over a period of time, will improve on the local control of the disease and also making the area more susceptible to the therapeutic and potentially curative effects of radiation.

The antifibrinolytic agent could be applied systemically, after surgical excision or could be injected where there is no excision into the cancerous area, such as via needle or catheter.

The inventive devices and methods are designed to improve cancer care by utilizing and improving the application of antifibrinolytic agents. One approach to the inventive treatment method would involve, surgical excision of the cancerous area, direct administration(such as via irrigation, needle injection or elution via a resorbable or non resorbable sponge) of an antifibrinolytic agent to the surgical area and/or the application of a pharmacologic preparation such as a hydrogel or resorbable wafer that would slowly release antifibrinolytic agents to retard the growth of the disease and concurrently make the area more radio sensitive over a period of time. The inventive methods and device could utilize the antifibrinolytic agent(s) that could be administered systemically (i.v. or oral) or directly (in open, endoscopic or topical procedures in its liquid injection form such as an irrigant or by needle injection) and/or via a time released or physically activated release (temperature, ph, or x-ray for example) of a preparation such as a biodegradable film a hydrogel sealant, biodegradable carrier such as, collagen, porcine gelatin, chitosan, fibrin, gellan gum or pectin or wafer (in similar fashion to Gliadel), liposomal drug delivery molecule or nano structured gels prior to surgical closure. Alternatively, or in addition to, antifibrinolytic drugs and preparations could be delivered by combining or constructing implanted devices such as transponding location beacons or rfd chips with coatings containing antifibrinolytics or other structures such as reservoirs that could release antifibrinolytic agents into the targeted area as well as via temporarily placed, biopsy cannulas (where the agent could be delivered prior to or post biopsy, for example, utilizing the needle cannula), catheters (similar to existing types that move csf for hydrocephalic patients) endovascular microcatheters (similar to those delivering embolic agents to aneurysms) and drug ports similar to morphine delivery for pain. Also, antifibrinolytic agents could be attached to molecules and strategies that are designed to seek out and attach themselves to cancerous tissue such as tumor fluorescence drugs. The device and method could be used during open surgical procedures such as tumor removal or debulking and/or delivered systemically and/or percutaneously and accurately (similar to the placement of deep brain stimulating electrodes), endovascular, via needle/cannula/catheter to preplanned (via historic image data or realtime images such as fluoroscopy, ultrasound, CT or MRI) clinical targets. Further, techniques that remove tissue such as biopsy or endoscopic debulking or place implants such as shunts for hydrocephalus or other objects into the body via needles or cannulas could also deliver antifibrinolytic agents before or after needle or cannula functions. Application of antifibrinolytic agents could be planned and optimized via surgical planning systems (which could predict the spread of the drug from a given insertion site by analyzing the targeted tissue via image data sets like MRI, as well as the molecular weight and diffusion characteristics of the delivered agent) and accurately delivered via real time imaging, image guided surgical navigation systems and stereotactic headframes and trajectory guides. Further, improved, inventive devices such as needles, cannulas and catheters (multi lumen and endovascular micro catheters for example) could be designed to best deliver the drug to the intended target while minimizing damage to healthy tissue that may occur during insertion.

This inventive method and device preparation centers on the innovative concept of improving cancer care by limiting the spread of cancerous/tumor cells by restraining fibrinolytic activity and increasing the radio sensitivity of the targeted area by placing antifibrinolytic agent(s) and/or related antifibrinolytic releasing devices such as catheters, needles and cannulas and/or drug releasing preparations, such as time released hydrogels, drug eluting device coatings, biodegradable films and gelatins (porcine for example) and polymers such as chitosan into the body systemically and/or directly to affect clinically relevant areas such as brain, prostate, lung, liver or breast.

It is to be understood that systemic delivery could be optimized to deliver the agents to affected areas via time released formats (extended release pills for example) as well as improved delivery via nano or optimized particles to allow more of the agent to reach the intended target such as crossing the blood brain barrier to reach brain tumors for example.

Although the invention has been particularly described with regard to impeding tumor growth and increasing radio sensitivity of cancerous tissue, the invention is not limited to those applications. For example, the invention could be used to deliver antifibrinolytic drugs to treat concussions (mild head trauma). In that regard, the same activity is at play wherein the trauma, whether from disease, injury or surgery, brings about the biological response of heightened fibrinolytic activity and inflammation. The invention would serve to counter this activity by the application of an antifibrinolytic agent.

What is claimed is:

1. In a method of impeding tumor growth and/or spread and increasing the radio sensitivity of cancerous tissue, the improvement being in administering an antifibrinolytic agent to the cancerous area of the patient to counter activation of a fibrinolytic process in that area.

2. The method of claim 1 wherein the antifibrinolytic agent is injected into the area before there is any excision of the area.

3. The method of claim 1 wherein there is a surgical excision of the cancerous area and then the antifibrinolytic agent is administered to the area.

4. The method of claim 3 wherein the antifibrinolytic agent is directly administered into the area by use of catheters, irrigation, needle injection or elution.

5. The method of claim 3 wherein a pharmacologic preparation is applied to the area to slowly release the antifibrinolytic agent.

6. The method of claim 1 wherein the antifibrinolytic agent is systemically or topically applied to the area.

7. The method of claim 3 wherein there is a time released or physically activated release of the antifibrinolytic agent to the area prior to and following surgical closure.

8. The method of claim 1 wherein the antifibrinolytic agent is applied by being implanted into the area.

9. The method of claim 1 wherein the antifibrinolytic agent is placed into the area by needle injection cannula (endoscopic or biopsy) or drug ports.

10. The method of claim 1 wherein the antifibrinolytic agent is attached to molecules and strategies that are designed to seek out and attach themselves to cancerous tissue.

11. The method of claim 1 wherein the antifibrinolytic agent is administered during open surgical procedures.

12. The method of claim 11 wherein the antifibrinolytic agent is delivered systemically and/or percutaneously and accurately to preplanned clinical targets.

13. The method of claim 1 wherein the antifibrinolytic agent is administered using techniques that visualize and/or remove tissue or place implants or other objects into the body via needles or cannulas before or after needle or cannula functions.

14. The method of claim 1 wherein the antifibrinolytic agent is applied by a plan optimized via surgical planning systems and accurately delivered via image guided surgical navigation systems, real time imaging, stereotactic head frames or trajectory guides.

15. In a method of treating concussions, the improvement being in administering an antifibrinolytic agent to the concussion area of the patient to counter activation of a fibrinolytic process in that area.
